(19) United States
(12) Patent Application Publication
(10) Pub. No.: US 2013/0289459 A1
(43) Pub. Date: Oct. 31, 2013

(54) CRANIAL HELMET FOR INFANTS

(71) Applicant: Gene P. BERNARDONI, Chicago, IL (US)

(72) Inventor: Gene P. BERNARDONI, Chicago, IL (US)

(21) Appl. No.: 13/455,627

(22) Filed: Apr. 25, 2012

(51) Int. Cl. A61F 5/00

(52) U.S. Cl. 602/17

(57) ABSTRACT

A helmet includes a generally rigid outer shell and an inner lining. The inner lining includes one or more layers comprising foam or another compressible material. The outer shell includes a slot to facilitate placement of the helmet onto and removal from a head of an infant. The slot defines first and second portions of the outer shell on either side thereof. The slot includes a convoluted or keyed segment that defines a projection in the first portion of the outer shell. The slot is shaped such that one or more edges of the projection engage one or more edges of a recess in the second portion, preventing separation of the first portion from the second portion along the slot. The helmet may include one or more pressure indicators configured to provide an indication of pressure exerted by a head of an infant wearing the helmet.

### BACKGROUND

The present disclosure relates generally to orthotic devices and, more specifically, to a cranial orthosis or helmet for an infant. More particularly, the present disclosure relates to a helmet configured to treat, prevent and/or inhibit plagiocephaly, which is a condition in which an infant's head has an uneven or irregular shape (e.g., a flat portion).

### SUMMARY

A helmet for an infant generally includes a generally rigid outer shell and an inner lining. The inner lining includes one or more layers made of foam and/or another compressible material. The outer shell includes a slot that is configured to facilitate placement of the helmet onto and removal from a head of an infant. The slot defines first and second portions of the outer shell on either side thereof. The slot includes a keyed segment that defines a projection in the first portion of the outer shell. The keyed segment also defines a recess in the second portion of the outer shell. The slot is shaped such that one or more edges of the projection engage one or more edges of the recess to prevent separation of the first portion from the second portion along the slot.

A helmet for an infant generally includes a generally rigid outer shell, an inner lining, and at least one pressure indicator. The inner lining includes one or more layers made of foam and/or another compressible material. The at least one pressure indicator is configured to provide an indication of pressure exerted on the indicator by a head of an infant wearing the helmet.

The foregoing is a summary and thus by necessity contains simplifications, generalizations, and omissions of detail. Consequently, those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a top plan view of a helmet according to an exemplary embodiment.

FIG. 2 is a rear plan view of the helmet depicted in FIG. 1.

FIG. 3 is a front plan view of the helmet depicted in FIG. 1.

FIG. 4 is a side plan view of the helmet depicted in FIG. 1, including a slot that defines a projection and a recess according to an exemplary embodiment.

FIG. 5 is a side plan view of a helmet depicted in FIG. 1, with the projection being disposed away from the recess.

FIG. 6 is a cross-sectional view of a portion of the helmet of FIG. 4 taken along line 6-6.

FIG. 7 is a cross-sectional view of a portion of the helmet of FIG. 1 and a sensor taken along line 7-7.

FIG. 8 is a cross-sectional view of a portion of the helmet of FIG. 1 and a pressure sensitive pad taken along line 8-8.

FIG. 9 is a block diagram of a helmet comprising an electronic sensor according to an exemplary embodiment.

### DETAILED DESCRIPTION

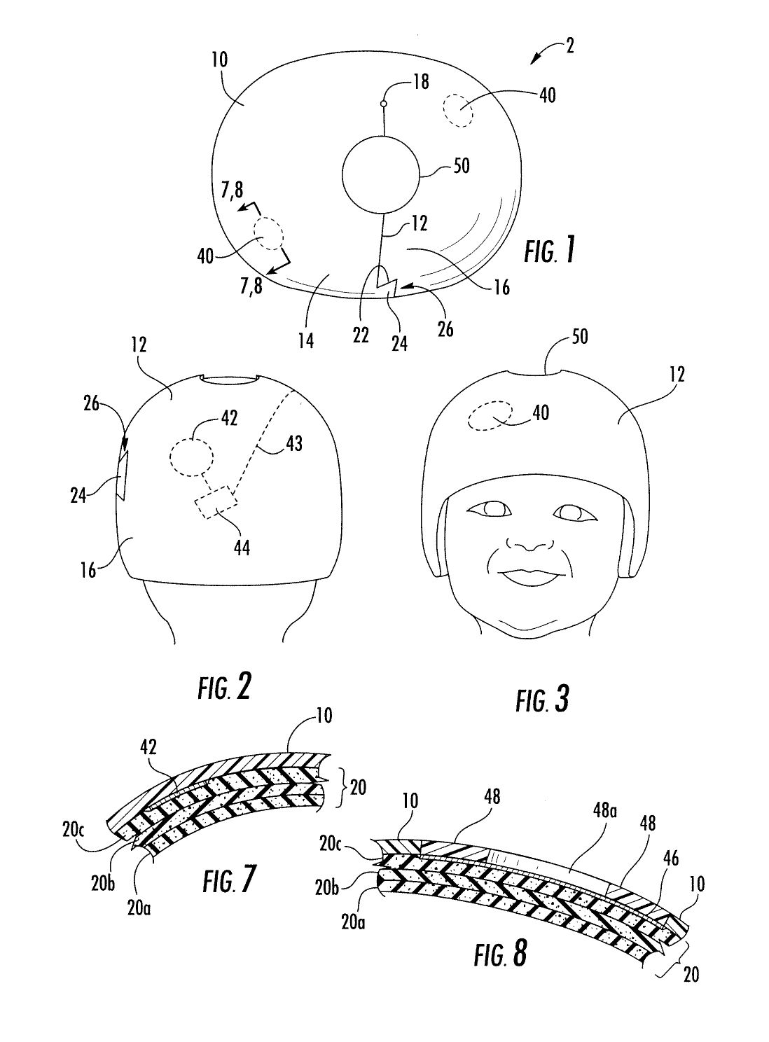

As shown generally in FIGS. 1-5, a helmet or cranial orthosis 2 is configured to treat, inhibit and/or prevent plagiocephaly, which is a condition in which an infant's head has an uneven or irregular shape (e.g., a flat portion). More particularly, the helmet 2 is configured to direct growth of the infant's head over time, so as to promote a proper or otherwise acceptable shape of the infant's head.

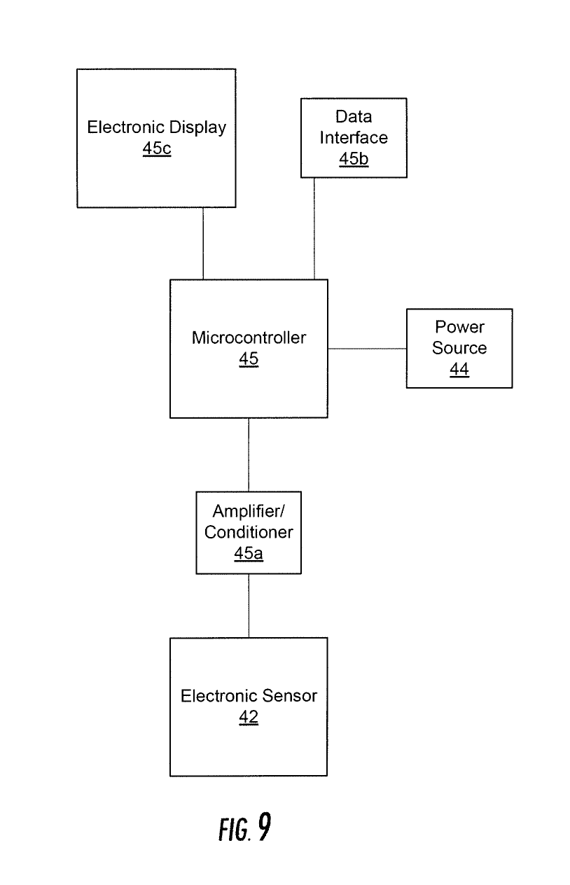

The helmet 2 generally includes an outer shell 10 and an inner lining 20. The helmet 2 may be fitted to an individual infant's head (e.g., to a model or casting of each infant's head) to include one or more voids (i.e., spaces or gaps) between the infant's head and the helmet 2. As the infant wears the helmet 2 over time (e.g., between approximately four and eight months in age), the helmet 2 will limit head growth in areas where the helmet 2 is in contact with the infant's head and will direct growth toward the voids. Furthermore, as shown in FIGS. 7 and 8, the inner lining 20 may comprise foam layers 20a, 20b, 20c (or layers made from another compressible material), which may be removed in succession to further accommodate overall growth of the infant's head.

The helmet 2 and its outer shell 10 may include various other features, such as an opening 12 having a convoluted or keyed segment 22, a latch or other type of closure mechanism 30, and/or one or more pressure sensors or pressure indicators 40. The helmet 2 may also include one or more venting apertures 50. Each of these features is discussed in further detail below.

The outer shell 10 is a generally rigid, plastic structure that is shaped according to an individual infant's head. In particular, the outer shell 10 is shaped such that the helmet 2 provides generally complete coverage of the infant's head, meaning that the helmet 2 has a generally closed upper end and comes in close proximity to the infant's ears. Complete coverage may advantageously provide for quicker treatment (i.e., correction of head shape) and/or prevent growth in unintended directions. The outer shell 10 may be formed, for example, by draping a heated thermoplastic sheet over a model resembling the infant's head (or the desired shape for the infant's head).

The opening 12 is configured, such that the helmet 2 may be placed onto or be removed from the infant's head. The opening 12 is provided in the form of a slot or cut in the outer shell 10 (as well as the underlying foam layers 20a, 20b, 20c). On either side of the slot 12 are first and second portions 14, 16, which are configured to normally engage, or be in close proximity to each other, at their edges and to be temporarily separated. In this manner, the helmet 2 may be expanded to facilitate placement or removal of the helmet 2. The slot 12 may be formed in the outer shell 10, for example, by cutting the outer shell 10 (after cooling) with a heated razor knife or in another manner that minimizes any gap formed in the slot 12 between the first and second portions 14, 16 of the outer shell 10.

The slot 12 extends generally upward from one side of the helmet 2. As shown in FIGS. 4 and 5, for example, the slot 12 may originate at a position generally above the infant's ear and may extend generally upward.

As shown in FIG. 1, the slot 12 may terminate, for example, at a position proximate the top of the helmet 2 (i.e., at the top of the infant's head) or at any other position suitable to allow for sufficient expansion of the helmet 2 for placement/removal, while also maintaining sufficient rigidity of the helmet 2 for treatment purposes. In one embodiment, the slot 12 extends past the top of the helmet 2 and terminates at a position on the opposite side from which the slot 12 originated. For example, the slot 12 may terminate at a position approximately one to two inches past the top of the helmet 2, or may terminate at a position approximately one to two inches past a venting aperture 50 positioned generally at the top of the helmet 2 (i.e., the slot 12 being interrupted by the venting aperture 50).

In other embodiments, the slot 12 may be configured in other manners including, for example, originating from a different position (e.g., another position along a side, front, or back portion of the helmet 2), extending in a different direction (e.g., sideways, convoluted, alone or in combination with extending upward), terminating at a different position (e.g., away from the top of the helmet 2), and the like.

The helmet 2 or outer shell 10 may also include an end feature 18 at the terminating position of the slot 12, which is configured to prevent unintended extension of the slot 12. For example, the end feature 18 may be configured to prevent tearing of the outer shell 10 as the helmet 2 is expanded along the slot 12 for placement/removal of the helmet. According to an exemplary embodiment, the end feature 18 is a round hole that may, for example, be drilled or otherwise created in the outer shell 10. Configured as a round hole, the end feature 18 functions to distribute forces (e.g., prevent localized stress) at the position at which the slot 12 terminates and thereby prevents deformation of the slot 12.

As shown in FIG. 4, the outer shell 10 also includes a convoluted or keyed segment or portion 22 (e.g., key) of the slot 12. The keyed segment 22 is configured to prevent sliding or shearing movement between the first and second portions 14, 16 of the outer shell 10. More particularly, the keyed segment 20 extends at an angle relative to the general direction of the slot 12, such that the first and second portions 14, 16 engage each other at their respective edges to prevent translational movement relative to each other in the general direction of the slot 12. Configured in this manner, the keyed segment 22 allows the slot 12 to behave as a generally rigid joint between the first and second portions 14, 16 of the outer shell 10 (e.g., for outer shell 10 to have strength of a continuous component). Advantageously, the slot 12 and keyed segment 22 provide for the outer shell 10 to be a fixed size throughout treatment of an infant, thus minimizing guess work and/or user error that might otherwise be associated with adjusting other devices used for similar purposes.

In one embodiment, the keyed segment 22 defines a projection or male portion 24 on the first portion 14 of the outer shell 10 and also defines a recess or female portion 26 on the second portion 16. The projection 24 and recess 26 are of complementary shape (e.g., being defined commonly by the slot 12), such that the recess 26 may receive the projection 24 therein. The projection 24 and recess 26 may be further configured to prevent separation of the first and second portions 14, 16 (i.e., prevent movement perpendicular to the general direction of the slot 12 and generally parallel with the surface of the outer shell 10).

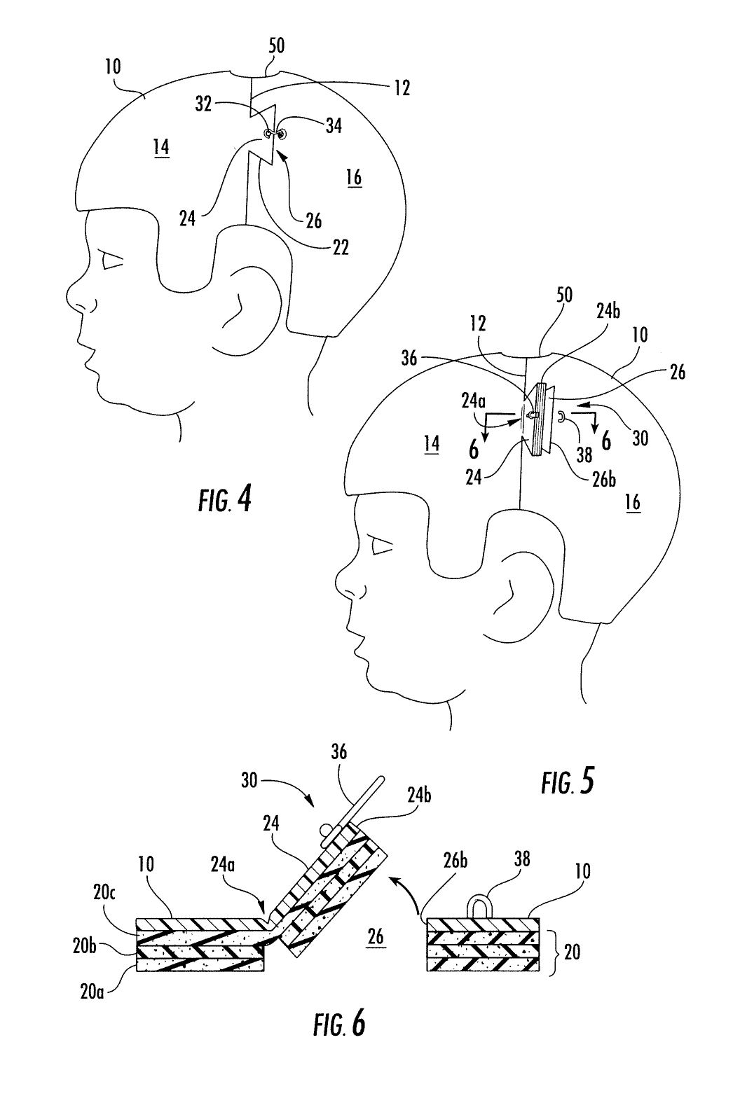

As shown in FIGS. 5 and 6, for example, the projection 24 extends outward from an inner portion 24a to an end portion 24b, wherein the inner portion 24a is narrower than the end portion 24b. Similarly, the recess 26 extends inward from an opening (not labeled) to an inner portion 26b, wherein the opening is narrower than the inner portion 26b. Shaped in this manner, the recess 26 is configured to receive the projection 24, and edges of the projection 24 engage edges of the recess 26. The relative widths (i.e., the inner portion 24a and opening being narrowing than the end portion 24b and inner portion 26b) prevent movement of the projection 24 relative to the recess 26 in a direction generally parallel with the surface of the outer shell 10. While the figures generally depict the keyed segment 22, and thereby the projection 24 and recess 26, having a trapezoidal shape, its shape may be any suitable geometry (e.g., diamond, rounded, etc.) as would be recognized by those skilled in the art.

The projection 24 is configured to be removed from the recess 26 in a direction generally perpendicular to the surface of the outer shell 12. For example, the inner portion 24a of the projection 24 may be configured as a hinge, so that the projection 24 may be folded or rotation outward to, thereby, be removed from the recess 26. For example, the outer shell 10 may be scored at the inner portion 24a of the projection 24, such that the inner portion 24a is weakened relative to adjacent portions of the outer shell 10 to define a hinge. According to other exemplary embodiments, the inner portion 24a may be configured in other manners to define a hinge including, for example, weakening the inner portion 24a in some other manner (e.g., forming with less material than adjacent portions), or providing the projection 24 as a separate component that is couple to the outer shell 10 with a mechanical hinge. Furthermore, the inner lining 20 may also be configured to define the hinge at the inner portion 24a of the projection 24. For example, one or more of the foam layers 20a, 20b, 20c may include a slit or be cut in a region proximate the inner portion 24a of the projection 24, so as to not prevent rotation of the projection 24 about the hinge. According to an exemplary embodiment, the two innermost layers 20a, 20b may include a slit, while the outer most layer 20c is continuous and does not include a slit.

The closure 30 is configured to prevent unintended removal of the projection 24 from the recess 26. The closure 30 bridges across the slot 12 to couple the second portion 16 of the outer shell 10 to the projection 24 on the first portion 14 of the outer shell 10. As shown in FIGS. 4 and 5, the closure 30 may comprise a hook 32 configured to engage a post 34, the hook 32 and post 34 each being positioned on one of the second portion 16 and the projection 24. According to the exemplary embodiment shown in FIG. 6, the closure 30 may comprise a slide 36 that is configured to slide into a hoop 38. According to still other exemplary embodiments, the closure 30 may be configured in other manners including, for example, being configured as a hook and loop fastener, a rotating hook with releasable latch for positively engaging a post, a side-release buckle with or without webbing, and the like. Advantageously, the combination of the slot 12 with the keyed segment 22 and closure 30 may suitably retain the helmet 2 on the infant's head obviating the need for a conventional strap (e.g., a helmet strap).

As shown in FIGS. 1-3, one or more pressure indicators or sensors 40 are configured to provide a user (e.g., a parent, doctor, or other treatment specialist) with an indication of the forces or pressures from the infant's head acting on the helmet 2. This information may be used, for example, to determine if the size or shape of the helmet needs to be adjusted based on changes in pressure readings (e.g., by removing one of the foam layers 20a, 20b, 20c to accommodate general growth of the infant's head).

In one embodiment, the one or more pressure indicators 40 may be positioned generally on an upper hemisphere of the helmet 2, a first pressure indicator 40 being biased to a forward, sideward position and another pressure indicator 40 being biased to a rearward, sideward position. According to other exemplary embodiments, the one or more pressure indicators 40 may be provided in other manners including, for example, being different in number (e.g., one, or more than two), being provided in different locations, and the like.

In the embodiment shown in FIGS. 2,7, and 9, each pressure indicator 40 comprises an electronic sensor 42 configured to measure or detect pressure applied thereto and to send corresponding signals to a microcontroller 45. As shown in the block diagram depicted in FIG. 9, leads 43 interconnect the electronic sensor 42, power source 44, microcontroller 45, an amplifier and/or signal conditioner 45a (if present), data interface 45b (if present), and/or a display 45c (if present).

Each electronic sensor 42 is positioned between the outer shell 10 and the outermost foam layer 20c (i.e., adjacent an inner surface of the outer shell 10), for example, each sensor 42 may be positioned on the outermost foam layer 20c prior to forming the outer shell 10 thereon (as described above). The electronic sensor 42 may, for example, be about 1″ in diameter and be sensitive up to about 25 lb. in force (or 110 N). The electronic sensor 42 may, for example, be a Tekscan brand sensor available under the trade name FlexiForce and having a model number of A401.

The power source 44 may, for example, be provided as a button cell battery 44 that is incorporated into the helmet 2 for powering the electronic sensor 42 (i.e., permanently or replaceably embedded into or underneath the outer shell 10). The power source may instead be remotely positioned to removably couple to the leads 43 for powering the electronic sensor 42 only when readings or measurements are to be taken.

The microcontroller 45, for example, may be incorporated into the helmet 2, or may be remotely positioned so as to be removably coupled to the leads 43 (e.g., via ports). The microcontroller 45 is configured to receive signals directly from the electronic sensor 42 (or by way of one or more sensor amplifiers or signal processers referred to generally as 45a), process signals received according to a stored software program executed by the microcontroller 45, and provide an indication of the pressure measured or detected by the electronic sensor 42 according to the signals received through the data interface 45b or display 45c. The microcontroller 45 may, for example, be (or include) a Microchip brand component having Part Number PIC16F886 available from Microchip Technology Inc. of Shanghai, China. The stored software program may, for example, be configured to convert readings from the electronic sensors 42 (e.g., voltage) into quantified units (e.g., psi), determine the sampling intervals at which readings from the electronic sensors 42 are taken, and the like.

The microcontroller 45 may be connected to a display 45c (e.g., an LCD display) for providing a real time indication or a numerical indication or value for of a pressure value (e.g., in pounds per square inch, or another unit that is a proxy to pressure, such as voltage). According to other exemplary embodiments, the microcontroller 45 may instead, or additionally, store or record the numerical indication (e.g., pressure values or proxies thereof) in memory (e.g., a non-volatile memory chip) at regular intervals, and a data interface 45b allows reading or downloading of the numerical indications from the memory.

In the embodiment shown in FIG. 8, each pressure indicator 40 comprises a pressure sensitive pad 46 that is configured to directly provide a visual indication of pressure applied thereto. For example, the pressure sensitive pad 46 may change between different colors and/or intensity of color as pressure applied thereto changes.

Similar to the electronic sensor 42, the pressure sensitive pad 46 is positioned between the outer shell 10 and the outermost foam layer 20c (i.e., adjacent the inner surface of the outer shell 10). For example, a plug or cutout 48 is removed from the outer shell 10 to allow placement and/or removal of the pressure sensitive pad 46 against the innermost foam layer 20c (i.e., from outside the helmet 2), and the cutout 48 is then replaced and/or recoupled to the outer shell 10, for example, with one or more closures similar to closure 30 (e.g., a hook and loop fastener, etc.). Configured in this manner, the cutout 48 is releasably coupled to the outer shell 10 so as to allow replacement of the pressure sensitive pad 46 (e.g., if the pressure sensitive pad 46 is disposable or configured for one-time use).

The pressure sensitive pad 46 may, for example, be a pressure indicating film available under the trade name Fuji Prescale from Sensor Products Inc. of Madison, N.J., USA. The pressure indicating film includes a donor sheet and a receiver sheet, the receiver sheet having a color forming material that reacts with a color-developing material from the receiver sheet, so as to change color or intensity of color (e.g., magenta) proportional to the amount of pressure being applied to the pressure sensitive pad 46. The donor sheet and receiver sheet are individually cut or otherwise formed to size for placement into the cutout 48. The pressure sensitive pad 46 and the cutout 48 may, for example, be approximately 1″ in diameter. The pressure sensitive pad 46 may have a pressure sensitive range of between approximately 28 and 85 psi, between approximately 70 and 350 psi, or any other suitable range.

The outer shell 10 also includes a visual access point 48a to provide visual access to the pressure sensitive pad 46 for reading the color or other visual indication of pressure. The visual access point 48a may, for example, be an aperture formed in the cutout 48. According to an exemplary embodiment, the cutout 48 has a diameter of approximately 1-1.5 inches (e.g., approximately 1.25 inches), and the aperture forming the visual access point 48a has a diameter of approximately 0.25-0.75 inches (e.g., approximately 0.5 inches).

In other embodiments, the pressure sensitive pad 46 and visual access point 48a may be configured in other manners including, for example, providing the pressure sensitive pad 46 from the interior of the helmet 2 (i.e., to avoid forming the cutout 48), providing the pressure sensitive pad 46 on the outermost foam layer 20c prior to forming the outer shell 10 thereon; providing the visual access point 48a as an aperture directly in the outer shell 10 (i.e., not as an aperture in the cutout 48), and/or providing the visual access point 48a as a solid window in the outer shell 10 (e.g., a clear plastic to provide for a continuous outer shell 10 in the region of the pressure sensitive pad 46).

The foregoing description of embodiments of the present invention have been presented for purposes of illustration and description. It is not intended to be exhaustive or to limit the present invention to the precise form disclosed, and modifications and variations are possible in light of the above teachings or may be acquired from practice of the present invention. The embodiments were chosen and described to explain the principles of the present invention and its practical application to enable one skilled in the art to utilize the present invention in various embodiments and with various modifications as are suited to the particular use contemplated.

What is claimed is:

1. A helmet for an infant, comprising:
a generally rigid outer shell; and
an inner lining comprising one or more layers of a compressible material, the inner lining operatively connected to an inner surface of the outer shell;
wherein the outer shell includes a slot configured to facilitate placement of the helmet onto and removal from a head of an infant, the slot defining first and second portions of the outer shell on either side thereof; and
wherein the slot includes a keyed segment defining a projection in the first portion of the outer shell and defining a recess in the second portion of the outer shell, wherein the slot is shaped such that one or more edges of the projection engage one or more edges of the recess to prevent separation of the first portion from the second portion along the slot.

2. The helmet of claim 1, wherein the slot is shaped to prevent separation of the first and second portion in a direction generally parallel with a surface of the outer shell adjacent the slot.

3. The helmet of claim 2, wherein the projection is configured to move in a direction generally perpendicular with the surface of the outer shell adjacent the slot, such that the projection may be removed from or received in the recess.

4. The helmet of claim 3 further comprising a closure, wherein the closure is configured to extend across the slot so as to couple the projection to the second portion of the outer shell to prevent removal of the projection from the recess.

5. The helmet of claim 1, wherein the helmet is configured to treat plagiocephaly.

6. A helmet for an infant, comprising:
a generally rigid outer shell;
an inner lining comprising one or more layers of a compressible material, the inner lining operatively connected to an inner surface of the outer shell; and
at least one pressure indicator operatively connected to at least one of the outer shell and the inner lining;
wherein the at least one pressure indicator is configured to provide an indication of pressure exerted by the helmet against a head of an infant wearing the helmet.

7. The helmet of claim 6, wherein the pressure indicator is disposed between the rigid outer shell and the inner lining.

8. The helmet of claim 7, wherein the pressure indicator comprises an electronic sensor that is configured to detect pressure and send a signal according to a detected pressure.

9. The helmet of claim 8, wherein a microcontroller is configured to receive the signal and process the signal to provide a numerical indication of the detected pressure.

10. The helmet of claim 9, wherein the microcontroller is configured to store a record of the numerical indication, and a data interface is configured to enable reading of the record of the numerical indication.

11. The helmet of claim 9, wherein the microcontroller is configured for the numerical indication to be displayed on an electronic display.

12. The helmet of claim 9, wherein the numerical indication is in units of pressure per square inch.

13. The helmet of claim 12, wherein the numerical indication is in units of voltage.

14. The helmet of claim 8, wherein the electronic sensor is sensitive to forces of up to about 25 pounds.

15. The helmet of claim 7, wherein the pressure indicator comprises a pressure sensitive material that is configured to provide a visual indication of pressure applied thereto.

16. The helmet of claim 15, wherein the pressure sensitive material is configured to change color in response to pressure applied thereto.

17. The helmet of claim 15, wherein the pressure sensitive material is configured to change intensity of color in response to pressure applied thereto.

18. The helmet of claim 15, wherein the outer shell includes a cutout that is removably coupled to the outer shell, and wherein the pressure sensitive material is positioned between the cutout and the inner foam lining.

19. The helmet of claim 15, wherein the outer shell includes a visual access point configured to allow viewing of the pressure sensitive material.

20. The helmet of claim 6, wherein the helmet is configured to treat plagiocephaly.
